(19) United States
(12) Patent Application Publication
(10) Pub. No.: US 2026/0042816 A1
(43) Pub. Date: Feb. 12, 2026

(54) TYROSINE KINASE INHIBITOR AND ACTIVIN TYPE 2 RECEPTOR ANTAGONIST COMBINATION THERAPY FOR TREATING PULMONARY ARTERIAL HYPERTENSION (PAH)

(71) Applicant: GB002, INC., San Diego, CA (US)

(72) Inventors: Ravi SITAPARA, San Diego, CA (US); Laura CARTER, San Diego, CA (US); Kristen Taylor MEADOWS, San Diego, CA (US)

(21) Appl. No.: 19/099,712

(22) PCT Filed: Aug. 10, 2023
(86) PCT No.: PCT/US2023/029993

§ 371 Date: Jan. 29, 2025

Related U.S. Application Data
(60) Provisional application No. 63/396,899, filed on Aug. 10, 2022.

(51) Int. Cl. C07K 14/71; A61K 9/00; A61K 31/497; A61K 38/00; A61P 9/12

(52) U.S. Cl. CPC C07K 14/71; A61K 9/0019; A61K 9/0073; A61K 31/497; A61P 9/12; A61K 38/00; C07K 2319/30

(57) ABSTRACT

Disclosed herein are kits and methods for treating pulmonary arterial hypertension (PAH), comprising administering to a subject in need thereof: •a therapeutically effective amount of a tyrosine kinase inhibitor or a pharmaceutically acceptable salt thereof; and•a therapeutically effective amount of a dimeric fusion protein comprising: •the extracellular domain of the activin type 2A (ACTR IIA) or the activin type 2B receptor (ACTR IIB); and the Fc domain of human immunoglobulin G1 (IgG1). In some embodiments, the tyrosine kinase inhibitor is Seralutinib or a pharmaceutically acceptable salt thereof and the fusion protein is Sotatercept.

### FIELD OF THE INVENTION

The present invention relates to tyrosine kinase inhibitors and dimeric fusion proteins, and their combined use in the treatment of diseases.

### BACKGROUND

Pulmonary hypertension (PH) is a chronic disorder affecting the small arteries in the lungs associated with high morbidity and mortality. The World Health Organization classifies PH into five groups based on the underlying associated disease:
Pulmonary arterial hypertension (PAH);
Pulmonary hypertension due to left heart disease;
Pulmonary hypertension due to lung diseases and/or hypoxia;
Chronic thromboembolic pulmonary hypertension (CTEPH); or
Pulmonary hypertension with other multifactorial mechanisms.

Pulmonary arterial hypertension (PAH) is a progressive disorder characterized by pulmonary vascular remodeling, resulting in high pulmonary artery pressure and progressive right ventricular dysfunction. The pathology of the disease includes plexiform lesions of disorganized angiogenesis and abnormal neointimal cellular proliferation, which obstruct blood flow through the pulmonary arterioles. PAH can be associated with several etiologies including familial forms and predisposing genetic abnormalities, such as genetic mutations in the bone morphogenetic type 2 receptor (BMPR2), endoglin, activin-like receptor kinase 1 (ALK1), mothers against decapentaplegic 9 (SMAD 9) and related pathways, autoimmune disorders (e.g., systemic sclerosis and scleroderma), congenital heart disease, liver disease with portal hypertension, and HIV infections.

The treatment of PAH was recently reviewed (“Pulmonary arterial hypertension: tailoring treatment to risk in the current era”, Gaine and McLaughlin, European Respiratory Review, 2017, 26, 170095). For most patients, combination therapy is the standard of care. This involves dual combination therapy with agents targeting the endothelin and nitric oxide pathways (e.g. a combination of an endothelin receptor antagonist (ERA) and phosphodiesterase-5 (PDE-5) inhibitor). Triple combination therapy with agents targeting the endothelin pathway, the nitric oxide pathway, and the prostacyclin (PGI2) pathway has also been used.

Despite these advances, PAH ultimately remains a fatal disease and efforts to slow disease progression are essential. While current treatments are effective in slowing disease progression, the 5-year survival rate is only 60%. There is therefore, a significant, urgent need for therapies for the disease.

### BRIEF SUMMARY

Described herein are methods for treating diseases, particularly pulmonary arterial hypertension (PAH), comprising administering to a subject in need thereof:
a therapeutically effective amount of a tyrosine kinase inhibitor or a pharmaceutically acceptable salt thereof; and
a therapeutically effective amount of a dimeric fusion protein comprising:
   the extracellular domain of the activin type 2A (ACTR IIA) or the activin type 2B receptor (ACTR IIB); and
   the Fc domain of human immunoglobulin G1 (IgG1).

In some embodiments, the tyrosine kinase inhibitor is a PDGF receptor inhibitor, a CSF1R receptor inhibitor, a c-KIT kinase inhibitor or a combination thereof. In some embodiments, the tyrosine kinase inhibitor is Seralutinib or a pharmaceutically acceptable salt thereof

In some embodiments, the dimeric fusion protein comprises the extracellular domain of the activin type 2A receptor. In other embodiments, the dimeric fusion protein comprises the extracellular domain of the activin type 2B receptor. In yet other embodiments, the dimeric fusion protein is Sotatercept.

In other embodiments, are methods for treating pulmonary arterial hypertension (PAH), comprising administering to a subject in need thereof, a therapeutically effective amount of Seralutinib or a pharmaceutically acceptable salt thereof; and a therapeutically effective amount of Sotatercept.

### SUMMARY OF FIGURES

FIG. 1 shows Seralutinib inhalation plus sotatercept injection shows more than additive efficacy in SuHx model of PAH. PAH was induced in animal by a administering a single injection SU5416 (20 mg/kg) on day 0, followed by exposure of animals to hypoxia (10% oxygen) for 21 days. SuHx rats were divided in four groups and treated with i) Control: Seralutinib vehicle inhalation+Sotatercept vehicle injection (grey bar) or ii) Seralutinib 15 mg/kg, by inhalation, twice daily+Sotatercept vehicle, subcutaneous injection, twice a week (bar with horizontal pattern) or iii) Sotatercept 5 mg/kg subcutaneous injection, twice a week+Seralutinib vehicle, by inhalation, twice daily (bar with vertical line pattern) or iv) Seralutinib 15 mg/kg, by inhalation, twice daily+Sotatercept 5 mg/kg subcutaneous injection, twice a week (white bar); normoxia animals were used a healthy controls. Bar graph showing individual normalized data point for (A) Right ventricular systolic pressure (RVSP) (FIG. 1A), (B) mean pulmonary artery pressure (mPAP) (FIG. 1B), (C) right ventricular hypertrophy measured by fulton's index (FIG. 1C), and PVR index (FIG. 1D).

FIG. 2 shows representative images of histological changes in lung by hematoxylin and eosin stain.

### DETAILED DESCRIPTION

As mentioned above, described herein is a method of treating pulmonary arterial hypertension (PAH), comprising administering to a subject in need thereof:
a therapeutically effective amount of a tyrosine kinase inhibitor or a pharmaceutically acceptable salt thereof; and
a therapeutically effective amount of a dimeric fusion protein comprising:
   the extracellular domain of the activin type 2A (ACTR IIA) or the activin type 2B receptor (ACTR IIB); and
   the Fc domain of human immunoglobulin G1 (IgG1).

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 25%, 20%, 15%, 10%, or 5% to a reference quantity, level, value, dimension, size, or amount.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

As used herein, a “subject” is a mammal, a bird, an aquatic animal such as a fish, or a reptile. In some embodiments, the subject is a human, a laboratory animal such as a mouse, rat or rabbit, a companion animal such as a dog or cat, a working animal such as a horse, donkey and the like, a livestock animal such as a cow, bull, pig, sheep, goat, deer, llama, alpaca and the like, or a captive wild animal such as those in zoos or wildlife parks including lions, leopards, cheetah, elephant, zebra, antelope, giraffe, koala, kangaroo and reptiles such as crocodiles, lizards, snakes and the like, a bird, especially a captive bird, such as a budgerigar or canary, cockatoo, parakeet, macaw, parrot and the like, or a fish, especially a captive fish such as tropical fish (zebra fish, guppy, Siamese fighting fish, clown fish, cardinal tetra and the like), dolphins, whales, and the like. In particular embodiments, the subject is a human.

The subject to be treated according to the methods described herein may be one who has been diagnosed with pulmonary hypertension, and in particular pulmonary arterial hypertension. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition. Further, it is understood that a subject to be treated according to the present disclosure may have been subjected to prior standard of care treatments, including monotherapy, dual therapy, triple therapy, or quadruple therapy. In particular embodiments, the subject who has been diagnosed with pulmonary hypertension, and in particular pulmonary arterial hypertension, is a human.

As used herein, the term “effective amount” or “therapeutically effective amount” refers to a quantity of a tyrosine kinase inhibitor and a dimeric fusion protein sufficient to achieve a desired effect in a subject being treated with those agents. Ideally, an effective amount is an amount sufficient to prevent or treat the disease without causing substantial toxicity in the subject. It is possible that the effective amount required for a tyrosine kinase inhibitor, either alone or in combination with other therapies that are not a dimeric fusion protein, is different to the effective amount required for a tyrosine kinase inhibitor in combination with a dimeric fusion protein. Likewise, it is possible that the effective amount required for a dimeric fusion protein either alone or in combination with other therapies that are not a tyrosine kinase inhibitor, is different to the effective amount required for a dimeric fusion protein in combination with a tyrosine kinase inhibitor. The effective amount will be dependent on the subject being treated, the severity of the disease, whether the subject has received prior treatment for the disease and the manner of administration of the agent. Methods of determining an effective amount of the disclosed therapies sufficient to achieve a desired effect in a subject will be understood by those of skill in the art.

The term “in combination with” as used herein refers to the tyrosine kinase inhibitor or a pharmaceutically acceptable salt thereof and the dimeric fusion protein being administered in a single composition, or separately, either simultaneously or sequentially. The tyrosine kinase inhibitor or a pharmaceutically acceptable salt thereof and the dimeric fusion protein may be administered at different times and different frequencies but in combination they exert biological effects at the same time or at overlapping times.

As used herein, “Seralutinib” refers to N-{3-[(1S)-1-{[6-(3,4-dimethoxyphenyl)pyrazin-2-yl]amino}ethyl]phenyl}-5-methylpyridine-3-carboxamide, also known as GB002 or PK10571, and is shown below:

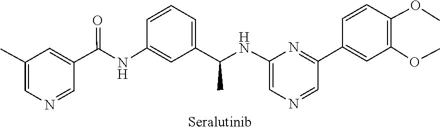

Seralutinib is a highly potent and selective inhibitor of PDGFRα and PDGFRβ, CSF1R, and c-KIT. An amorphous form of Seralutinib is described in U.S. Pat. Nos. 9,815,815 and 10,231,966. Formulations comprising Seralutinib are described in U.S. Pat. No. 9,925,184 and published US patent application US 2021/0038510. Combinations comprising Seralutinib are described in U.S. Pat. Nos. 10,231,966 and 11,364,238.

Inhaled seralutinib was an effective treatment of severe PAH in two animal models, with improved cardiopulmonary hemodynamics, reduction in NT-proBNP, reverse remodeling of pulmonary vascular pathology, and improvement in inflammatory biomarkers. Seralutinib showed greater efficacy compared to imatinib in a preclinical study (see “Inhaled Seralutinib Exhibits Potent Efficacy in Models of Pulmonary Arterial Hypertension”, Galkin et. al., European Respiratory Journal, 2022). A Phase 2 randomized, double-blind, placebo-controlled trial, evaluating the efficacy and safety of inhaled seralutinib in subjects with WHO Group 1 Pulmonary Hypertension is ongoing.

As used herein, “Sotatercept” refers to a soluble fusion protein composed of the extracellular domain of the activin receptor type IIA (ActR IIA) linked to the Fc portion of human IgG1 with anabolic bone activity. Sotatercept traps multiple members of the TGF-β superfamily, including activins and growth differentiation factors. Mutations in bone morphogenetic protein receptor type 2 (BMPR2), a member of the transforming growth factor β (TGF-β) superfamily, are a major factor underlying heritable PAH. BMPR2 is important in maintaining endothelial integrity in pulmonary arteries. Mutations that reduce signaling in the BMPR-II pathway promote endothelial dysfunction, increased cellular proliferation and pulmonary vascular remodeling. Sotatercept restores balance between the growth-promoting activin growth differentiation factor pathway and the growth-inhibiting BMP pathway.

Therapeutic treatment with a rodent analog of Sotatercept (RAP-011) reverses vascular modeling in rats with PAH. In a phase 2 clinical study, beneficial effects of Sotatercept were reported in PAH patients treated with standard-of-care therapies. In 2019 the FDA designated Sotatercept an orphan drug for the treatment of pulmonary arterial hypertension (PAH). In 2020 Sotatercept received a breakthrough therapy designation from the FDA and was granted a priority medicines designation by the European Medicines Agency (EMA), for the same indication.

### Methods of Treating PAH

In one embodiment, a method of treating pulmonary arterial hypertension (PAH) is presented, comprising administering to a subject in need thereof:
a therapeutically effective amount of a tyrosine kinase inhibitor or a pharmaceutically acceptable salt thereof; and
a therapeutically effective amount of a dimeric fusion protein comprising:
   the extracellular domain of the activin type 2A (ACTR IIA) or the activin type 2B receptor (ACTR IIB); and
   the Fc domain of human immunoglobulin G1 (IgG1).

Receptor tyrosine kinases (RTK) or tyrosine kinase receptors (TKR) are polypeptides that regulate the regeneration, remodeling, development, and differentiation of cells. They are high-affinity cell surface receptors for many polypeptide growth factors, cytokines, and hormones. Mutations in receptor tyrosine kinases lead to activation of a series of signaling cascades which in turn effects protein expression. Receptor tyrosine kinases are part of the larger family of protein tyrosine kinases, encompassing the receptor tyrosine kinase proteins which contain a transmembrane domain, and the non-receptor tyrosine kinases which lack transmembrane domains. Approximately 20 different RTK classes have been identified including the EGF (or ErbB), Insulin, PDGF, VEGF, FGF, CCK, NGF, HGF, Eph, AXL, TIE, RYK, DDR, RET, ROS, LTK, ROR, and MuSK receptor families.

Platelet-derived growth factor receptors (PDGF-R) are tyrosine kinase receptors for members of the platelet-derived growth factor (PDGF) family. PDGF subunits-A and -B are important factors regulating cell proliferation, cellular differentiation, cell growth, and development. There are two forms of the PDGF-R, alpha and beta each encoded by a different gene. The platelet derived growth factor receptor (PDGFR) is associated with pulmonary diseases, tissue fibrosis and solid tumors.

In one embodiment, the tyrosine kinase inhibitor of the method of treating PAH is a PDGF receptor inhibitor or a pharmaceutically acceptable salt thereof, a CSF1R receptor inhibitor or a pharmaceutically acceptable salt thereof, a c-KIT kinase inhibitor or a pharmaceutically acceptable salt thereof, or a combination thereof. In some embodiments, the tyrosine kinase inhibitor is Acalabrutinib or a pharmaceutically acceptable salt thereof, Afatinib or a pharmaceutically acceptable salt thereof, Alectinib or a pharmaceutically acceptable salt thereof, Avapritinib or a pharmaceutically acceptable salt thereof, Axitinib (Inlyta®) or a pharmaceutically acceptable salt thereof, Baricitinib or a pharmaceutically acceptable salt thereof, Binimetinib or a pharmaceutically acceptable salt thereof, Bosutinib (Bosulif®) or a pharmaceutically acceptable salt thereof, Brigatinib or a pharmaceutically acceptable salt thereof, Cabozantinib or a pharmaceutically acceptable salt thereof, Capmatinib or a pharmaceutically acceptable salt thereof, Ceritinib or a pharmaceutically acceptable salt thereof, Cobimetinib or a pharmaceutically acceptable salt thereof, Crizotinib or a pharmaceutically acceptable salt thereof, Dacomitinib or a pharmaceutically acceptable salt thereof, Entrectinib or a pharmaceutically acceptable salt thereof, Erdafitinib or a pharmaceutically acceptable salt thereof, Erlotinib (Tarceva®) or a pharmaceutically acceptable salt thereof, Fedratinib or a pharmaceutically acceptable salt thereof, Fostamatinib or a pharmaceutically acceptable salt thereof, Gefitinib or a pharmaceutically acceptable salt thereof, Gilteritinib or a pharmaceutically acceptable salt thereof, Ibrutinib or a pharmaceutically acceptable salt thereof, Imatinib (Gleevec®) or a pharmaceutically acceptable salt thereof, Lapatinib or a pharmaceutically acceptable salt thereof, Larotrectinib or a pharmaceutically acceptable salt thereof, Lenvatinib or a pharmaceutically acceptable salt thereof, Lorlatinib or a pharmaceutically acceptable salt thereof, Midostaurin or a pharmaceutically acceptable salt thereof, Neratinib (Tasigna®) or a pharmaceutically acceptable salt thereof, Osimertinib or a pharmaceutically acceptable salt thereof, Pazopanib (Votrient®) or a pharmaceutically acceptable salt thereof, Pemigatinib or a pharmaceutically acceptable salt thereof, Pexidartinib or a pharmaceutically acceptable salt thereof, Ponatinib or a pharmaceutically acceptable salt thereof, Regorafenib or a pharmaceutically acceptable salt thereof, Ripretinib or a pharmaceutically acceptable salt thereof, Ruxolitinib or a pharmaceutically acceptable salt thereof, Selpercatinib or a pharmaceutically acceptable salt thereof, Selumetinib or a pharmaceutically acceptable salt thereof, Seralutinib or a pharmaceutically acceptable salt thereof, Sorafenib or a pharmaceutically acceptable salt thereof, Sunitinib (Sutent®) or a pharmaceutically acceptable salt thereof, Tofacitinib or a pharmaceutically acceptable salt thereof, Trametinib or a pharmaceutically acceptable salt thereof, Tucatinib or a pharmaceutically acceptable salt thereof, Upadacitinib or a pharmaceutically acceptable salt thereof, Vandetanib or a pharmaceutically acceptable salt thereof, Zanubrutinib or a pharmaceutically acceptable salt thereof, or a combination thereof.

In some embodiments, the tyrosine kinase inhibitor is Seralutinib or a pharmaceutically acceptable salt thereof. In some embodiments, the tyrosine kinase inhibitor is Seralutinib. In some embodiments, the tyrosine kinase inhibitor is a pharmaceutically acceptable salt of Seralutinib.

In some embodiments, the dimeric fusion protein comprises the extracellular domain of the activin type 2A receptor. In other embodiments, the dimeric fusion protein comprises the extracellular domain of the activin type 2B receptor. In other embodiments, the dimeric fusion protein is Sotatercept.

Further described herein is a method of treating pulmonary arterial hypertension (PAH), comprising administering to a subject in need thereof:
a therapeutically effective amount of Seralutinib or a pharmaceutically acceptable salt thereof; and
a therapeutically effective amount of Sotatercept.

The combination therapy described herein is intended to embrace the administration of the tyrosine kinase inhibitor or a pharmaceutically acceptable salt thereof and a dimeric fusion protein as both a stand-alone dual combination therapy, as well as in further combination with other biologically active ingredients as well as non-drug therapies (e.g., holistic therapies). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In some embodiments, the subject is receiving stable background therapy for pulmonary arterial hypertension. In some embodiments, the stable background therapy is monotherapy, double therapy, or triple therapy. In other embodiments, the stable background therapy is monotherapy. In other embodiments, the stable background therapy is double therapy. In other embodiments, the stable background therapy is triple therapy. In some embodiments, the stable background therapy comprises an endothelin-receptor antagonist, a phosphodiesterase-5 (PDE-5) inhibitor, a prostacyclin analogue, a prostacyclin-receptor agonist, a soluble guanylate cyclase stimulator, or a combination thereof. In some embodiments, the stable background therapy comprises an endothelin-receptor antagonist. In some embodiments, the stable background therapy comprises a phosphodiesterase-5 (PDE-5) inhibitor. In some embodiments, the stable background therapy comprises a prostacyclin analogue. In some embodiments, the stable background therapy comprises a soluble guanylate cyclase stimulator. In some embodiments, the stable background therapy comprises a combination of at least two therapies selected from an endothelin-receptor antagonist, a phosphodiesterase-5 (PDE-5) inhibitor, a prostacyclin analogue, a prostacyclin-receptor agonist.

In some embodiments, the method further comprises administering to the subject an endothelin-receptor antagonist, a phosphodiesterase type 5 (PDE-5) inhibitor, a prostacyclin analogue, a prostacyclin receptor agonist, a soluble guanylate cyclase stimulator, or a combination thereof.

In some embodiments, the method further comprises administering to the subject a therapeutically effective amount of an endothelin receptor agonist. In some embodiments, the endothelin receptor agonist is Ambrisentan (Letairis®), Macitentan (OPSUMIT®), or Bosentan.

In some embodiments, the method further comprises administering to the subject a therapeutically effective amount of a phosphodiesterase type 5 (PDE-5) inhibitor. In some embodiments, the phosphodiesterase type 5 (PDE-5) inhibitor is sildenafil, tadalafil, vardenafil, avanafil, or udenafil.

In some embodiments, the method further comprises administering to the subject a prostacyclin analogue. In some embodiments, the prostacyclin receptor analogue is epoprostenol, treprostinil, iloprost, or beraprost.

In some embodiments, the method further comprises administering to the subject a prostacyclin receptor agonist. In some embodiments, the prostacyclin receptor agonist is selexipag, or ralinepag.

In some embodiments, the method further comprises administering to the subject a soluble guanylate cyclase stimulator. In some embodiments, the soluble guanylate cyclase stimulator is riociguat, or vericiguat.

In some embodiments, the method further comprises administering to the subject tadalafil, selexipag, ralinepag, or combinations thereof. In other embodiments, the method further comprises administering to the subject tadalafil, selexipag, or combinations thereof.

In some embodiments of the method of treating PAH, the PAH is mild or moderate PAH. In some embodiments, the PAH is mild PAH. In some embodiments, the PAH is moderate PAH. In some embodiments, the method reduces a morbidity risk and a mortality risk of PAH. In some embodiments, the method reduces a morbidity risk of the PAH. In some embodiments, the method reduces a mortality risk of the PAH. In some embodiments, the method reduces a morbidity risk, a mortality risk, or both, of the PAH.

In some embodiments of the present invention, the tyrosine kinase inhibitor is administered by inhalation. In some embodiments, Seralutinib or a pharmaceutically acceptable salt thereof, is administered by inhalation. In some embodiments, Seralutinib or a pharmaceutically acceptable salt thereof, is administered by inhalation with a dry powder inhaler. In some embodiments, Seralutinib or a pharmaceutically acceptable salt thereof, is administered once daily or twice daily. In some embodiments, Seralutinib or a pharmaceutically acceptable salt thereof, is administered once. In some embodiments, Seralutinib or a pharmaceutically acceptable salt thereof, is administered twice daily. In some embodiments, Seralutinib or a pharmaceutically acceptable salt thereof, is administered three times a day.

In some embodiments, the dimeric fusion protein is administered by injection. In some embodiments, Sotatercept is administered by injection. In some embodiments, Sotatercept is administered by subcutaneous injection. In some embodiments, Sotatercept is administered once a week, once every two weeks, once every three weeks, or once a month. In other embodiments, Sotatercept is administered once a week. In some embodiments, Sotatercept is administered once every two weeks. In some embodiments, Sotatercept is administered every three weeks. In some embodiments, Sotatercept is administered once a month.

### Dosage Regimens

Therapeutic agents described herein can be administered before, during, or after the occurrence or diagnosis of a disease, and the timing of administering the agents can vary. For example, the agents can be used as a prophylactic and can be administered to subjects with a propensity to particular diseases in order to lessen a likelihood of the occurrence of the disease. The agents can be administered to a subject during or as soon as possible after the onset of the symptoms. The initial administration can be via any route practical. A therapeutic agent can be administered as soon as is practicable after the onset of a disease or condition is detected or suspected, and for a length of time necessary for the treatment of the disease. The length of treatment can vary for each subject.

In some embodiments, the tyrosine kinase inhibitor or a pharmaceutically acceptable salt thereof is administered to the subject in need thereof separately from the dimeric fusion protein.

In some embodiments, the tyrosine kinase inhibitor or a pharmaceutically acceptable salt thereof is administered by inhalation. In some embodiments, Seralutinib or a pharmaceutically acceptable salt thereof is administered by inhalation.

In some embodiments, the tyrosine kinase inhibitor or a pharmaceutically acceptable salt thereof is administered once daily or twice daily. In some embodiments, Seralutinib or a pharmaceutically acceptable salt thereof is administered once daily. In other embodiments, Seralutinib or a pharmaceutically acceptable salt thereof is administered twice daily.

In some embodiments, the dimeric fusion protein is administered by injection. In some embodiments, the dimeric fusion protein is administered by subcutaneous injection. In some embodiments, Sotatercept is administered by injection. In some embodiments, Sotatercept is administered by subcutaneous injection.

In some embodiments, the dimeric fusion protein is administered twice a week, once a week, once every two weeks, once every three weeks, or once a month. In some embodiments, Sotatercept is administered twice a week, once a week, once every two weeks, once every three weeks, or once a month. In some embodiments, Sotatercept is administered twice a week. In other embodiments, Sotatercept is administered once a week. In other embodiments, Sotatercept is administered once every two weeks. In other embodiments, Sotatercept is administered once every three weeks. In other embodiments, Sotatercept is administered once a month.

In some embodiments, the tyrosine kinase inhibitor or a pharmaceutically acceptable salt thereof is administered at least once, prior to, simultaneously, or sequentially with the dimeric fusion protein. In particular embodiments, multiple doses of the tyrosine kinase inhibitor or a pharmaceutically acceptable salt thereof are administered over a period of time beginning before or together with administration of the dimeric fusion protein and then continuing after administration of the dimeric fusion protein.

In some embodiments, the tyrosine kinase inhibitor or a pharmaceutically acceptable salt thereof is administered more than once and on a regular basis before, simultaneously, and after administration of the dimeric fusion protein. In some embodiments, the tyrosine kinase inhibitor or a pharmaceutically acceptable salt thereof is administered before administration of the dimeric fusion protein. In other embodiments, the tyrosine kinase inhibitor or a pharmaceutically acceptable salt thereof is administered simultaneously or sequentially with the administration of the dimeric fusion protein, and at least once subsequently to administration of the dimeric fusion protein. The tyrosine kinase inhibitor or a pharmaceutically acceptable salt thereof may be administered 1 week to 1 day prior to administration of the dimeric fusion protein, especially 1 to 3 days before the administration of the dimeric fusion protein. The tyrosine kinase inhibitor or a pharmaceutically acceptable salt thereof may be administered simultaneously or sequentially with the dimeric fusion protein, either immediately before or immediately after the administration of the dimeric fusion protein. The tyrosine kinase inhibitor or a pharmaceutically acceptable salt thereof may also be administered one or more times over the month after administration of the dimeric fusion protein, for example, once a week, once every 5 days, once every 4 days, once every 3 days, once every 2 days, once every day, or twice every day, especially once or twice every day. Subsequent administration of the tyrosine kinase inhibitor or a pharmaceutically acceptable salt thereof may continue such that 1 to 10 doses of the tyrosine kinase inhibitor or a pharmaceutically acceptable salt thereof are administered after administration of the dimeric fusion protein, especially 1 to 70 doses, 1 to 60 doses, 1 to 50 doses, 1 to 40 doses, 1 to 30 doses, 1 to 20 doses, 1 to 10 doses, 1 to 8 doses, 1 to 6 doses, 1 to 4 doses, or 1 to 2 doses.

The dimeric fusion protein is administered in an effective amount. An “effective amount” means an amount necessary to at least partly attain the desired therapeutic response. The amount varies depending upon the health and physical condition of the individual to be treated, the taxonomic pulmonary hypertension group of the individual to be treated, the formulation of the composition, the severity of the pulmonary hypertension, the assessment of the medical situation, and other relevant factors. It is expected that the amount will fall in a relatively broad range. An effective amount, for example, may lie in the range of about 0.1 ng per kg of body weight to 1 g per kg of body weight per dosage. The dosage is preferably in the range of 1 μg to 0.5 g per kg of body weight per dosage, such as is in the range of 0.1 mg to 100 mg per kg of body weight per dosage, 1 mg to 25 mg per kg of body weight per dosage, or 5 mg per kg of body weight per dosage. In some embodiments, where the dimeric fusion protein dosage is administered by injection subcutaneously, the dosage is in the range of 0.1 mg to 25 mg per kg of body weight, for example 1 mg to 10 mg per kg of body weight, such as 1 mg/kg of body weight, 2 mg/kg of body weight, 3 mg/kg of body weight, 4 mg/kg of body weight, or 5 mg/kg of body weight. Dosage regimes may be adjusted to provide the optimum therapeutic response. For example, in some embodiments, where administration of the dimeric fusion protein is by subcutaneous injection, the dimeric fusion protein is administered once a month and the progress of treatment monitored. In other embodiments, the dimeric fusion protein is administered once every three weeks and the progress of treatment monitored. In other embodiments, the dimeric fusion protein is administered once every two weeks and the progress of treatment monitored. In other embodiments, the dimeric fusion protein is administered once every week and the progress of treatment monitored. In other embodiments, the dimeric fusion protein is administered twice a week and the progress of treatment monitored.

The tyrosine kinase inhibitor or a pharmaceutically acceptable salt thereof may also be administered in an effective amount. Again, the amount of the tyrosine kinase inhibitor or a pharmaceutically acceptable salt thereof considered to be effective will depend on the health and physical condition of the individual to be treated, the taxonomic pulmonary hypertension group of the individual to be treated, the formulation of the composition, the severity of the pulmonary hypertension, the assessment of the medical situation, and other relevant factors. It is expected that the amount of the tyrosine kinase inhibitor or a pharmaceutically acceptable salt thereof will fall within a fairly broad range of amounts. An effective amount may lie in the range of about 0.1 ng per kg to about 500 mg per kg body weight per dosage. The dosage is preferably in the range of 100 μg to 100 mg per kg of body weight per dosage, 1 mg to 50 mg per kg of body weight per dosage, 1 mg to 20 mg per kg of body weight per dosage, or 5 mg to 15 mg per kg of body weight per dosage. In some embodiments, where the dosage of the tyrosine kinase inhibitor or pharmaceutically acceptable salt thereof is administered by inhalation, the dosage is in the range of 1 mg to 100 mg per kg of body weight, for example 5 mg to 25 mg per kg of body weight, such as 5 mg/kg of body weight, 10 mg/kg of body weight, 15 mg/kg of body weight, 20 mg/kg of body weight, or 25 mg/kg of body weight. Dosage regimes may be adjusted to provide the optimum therapeutic response. For example, in some embodiments, where administration of the tyrosine kinase inhibitor of a pharmaceutically acceptable salt thereof is by inhalation, the tyrosine kinase inhibitor of a pharmaceutically acceptable salt thereof is administered twice a day and the progress of treatment monitored. In other embodiments, the tyrosine kinase inhibitor of a pharmaceutically acceptable salt thereof is administered once a day and the progress of treatment monitored. In other embodiments, the tyrosine kinase inhibitor of a pharmaceutically acceptable salt thereof is administered once every two days and the progress of treatment monitored. In other embodiments, the tyrosine kinase inhibitor of a pharmaceutically acceptable salt thereof is administered once every three days and the progress of treatment monitored. In other embodiments, the tyrosine kinase inhibitor of a pharmaceutically acceptable salt thereof is administered once every five days and the progress of treatment monitored. In other embodiments, the tyrosine kinase inhibitor of a pharmaceutically acceptable salt thereof is administered once every six days and the progress of treatment monitored. In other embodiments, the tyrosine kinase inhibitor of a pharmaceutically acceptable salt thereof is administered once every week and the progress of treatment monitored.

### Kits

The compositions of a tyrosine kinase inhibitor or a pharmaceutically acceptable salt thereof and a dimeric fusion protein may be formulated separately and sold together in a kit or package. In one embodiment, each kit may comprise one or more doses of each compound useful in the treatment or prevention of PAH. In another embodiment, each kit may comprise one or more containers. In a further embodiment, each container of the kit may contain one or more doses of one or more compounds useful in the treatment or prevention of PAH. In one embodiment, each container of the kit contains one or more doses of a different compound useful in the treatment or prevention of PAH.

In one embodiment is presented a kit useful in the treatment or prevention of pulmonary arterial hypertension (PAH) comprising one or more containers containing:
a tyrosine kinase inhibitor or a pharmaceutically acceptable salt thereof; and
a dimeric fusion protein comprising:
   the extracellular domain of the activin type 2A (ACTR IIA) or the activin type 2B receptor (ACTR IIB); and
   the Fc domain of human immunoglobulin G1 (IgG1).

In one embodiment, at least one container of the kit contains a tyrosine kinase inhibitor or a pharmaceutically acceptable salt thereof. In a preferred embodiment, the tyrosine kinase inhibitor or a pharmaceutically acceptable salt thereof is Seralutinib or a pharmaceutically acceptable salt thereof. In another embodiment, at least one container of the kit contains the dimeric fusion protein. In a preferred embodiment, the dimeric fusion protein is Sotatercept. In one embodiment, the kit comprises at least one container containing Seralutinib or a pharmaceutically acceptable salt thereof and at least one separate container containing Sotatercept.

In one embodiment is presented a kit comprising:
one or more doses of a therapeutically effective amount of a tyrosine kinase inhibitor or a pharmaceutically acceptable salt thereof; and
one or more doses of a therapeutically effective amount of a dimeric fusion protein comprising:
   the extracellular domain of the activin type 2A (ACTR IIA) or the activin type 2B receptor (ACTR IIB); and
   the Fc domain of human immunoglobulin G1 (IgG1).

In some embodiments, the tyrosine kinase inhibitor or a pharmaceutically acceptable salt thereof is formulated for administration by inhalation. In one embodiment, the tyrosine kinase inhibitor or a pharmaceutically acceptable salt thereof is formulated as a dry powder for administration by inhalation with a dry powder inhaler. In some embodiments, the kit comprises one or more doses of the tyrosine kinase inhibitor or a pharmaceutically acceptable salt thereof and a dry powder inhaler.

In some embodiments, the dimeric fusion protein is formulated for subcutaneous injection in a single bolus dose or in a multiple dose form. For example, the kit may contain the dimeric fusion protein in a pre-filled syringe, as a liquid in a vial ready for uptake into a syringe, or as a solid ready for dissolution before uptake into a syringe. The liquid or solid formulations may be single dose formulations or multiple dose formulations. In other embodiments, the kit may contain multiple doses of the dimeric fusion protein each formulated separately in a prefilled syringe, as a liquid in a vial ready for uptake into a syringe, or as a solid ready for dissolution and uptake into a syringe.

The kit may further comprise one or more of various pharmaceutical kit components, such as, for example, containers with one or more pharmaceutically acceptable carriers. The kit may further comprise an insert or a label with instructions for use of each formulation. The insert or label may further comprise how to prepare each dosage form if required, including quantities of the components to be administered and/or guidelines for mixing the components, how to administer each dosage, and/or when to administer each dosage.

### EXAMPLES

The following examples are provided for illustrative purposes only and not to limit the scope of the claims provided herein. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1: PAH Rat Model

SuHx-mediated (SU5416 plus hypoxia) pulmonary hypertension was induced in male Sprague Dawley rats (see “Inhibition of the VEGF receptor 2 combined with chronic hypoxia causes cell death-dependent pulmonary endothelial cell proliferation and severe pulmonary hypertension”, Taraseviciene-Stewart et. al., FASEB J, 2001, 15, 427-438 and “TORREY, a Phase 2 study to evaluate the efficacy and safety of inhaled seralutinib for the treatment of pulmonary arterial hypertension”, Frantz R P, et. al., Pulmonary Circulation 2021; 11(4), 1-7). Semaxanib (SU5416) was administered to rats weighing 200-250 g as a single subcutaneous injection (20 mg/kg). Rats were housed for 3 weeks in 10% oxygen and subsequently re-exposed to normoxia during 4-week treatment period.

The rats were divided into 5 treatment groups as summarized in Table 1.

TABLE 1

| | | |
|---|---|---|
| GROUP 1 | Healthy | (no disease state induction) |
| GROUP 2 | Control | Seralutinib vehicle + Sotatercept vehicle |
| GROUP 3 | Administered | Seralutinib 15 mg/kg, by inhalation, twice |
| | | daily + Sotatercept vehicle, subcutaneous |
| | | injection, twice a week |
| GROUP 4 | Administered | Sotatercept 5 mg/kg subcutaneous injection, |
| | | twice a week + Seralutinib vehicle, by |
| | | inhalation, twice daily |
| GROUP 5 | Administered | Seralutinib 15 mg/kg, by inhalation, twice |
| | | daily + Sotatercept 5 mg/kg subcutaneous |
| | | injection, twice a week |

Rats underwent treatment for four weeks as described above, with treatment starting on day 22, after disease induction. After 4 weeks of treatment, the following parameters were evaluated: right ventricular systolic pressure (RVSP); Fulton index (weight ratio of right ventricle to the left ventricle and septum); and mean pulmonary arterial pressure (mPAP).

For each of the measured parameters (RVSP, Fulton's Index and mPAP), the combination of Seralutinib+Sotatercept (group 5) resulted in a significant decrease versus: Control (group 2, Seralutinib Vehicle Inhalation+Sotatercept vehicle, subcutaneous injection); Seralutinib treatment alone (group 3); and Sotatercept treatment alone (group 4).

In each instance, the observed effect was synergistic for the combination over the additive effect of agents alone. Furthermore, 3 out of 7 animals from the Seralutinib+Sotatercept combination treatment group (Group 5), presented RVSP levels that had dropped to the same levels as the healthy animals (group 1). More specific details concerning this study are presented below.

### Induction of PAH

Animals from (Group 2-5) received a single subcutaneous injection of SU5416 (20 mg/kg) on Day 0 and exposed to hypoxia (10% O₂) for 21 days followed by 4 weeks of normoxia. Group 1 animals (healthy group) remained in cages exposed to ambient oxygen (normoxic) levels for 49 days. Food and water were given ad libitum. The animals were randomized on Day 21 among treatment groups based on their body weight and the results of transthoracic echocardiography.

Treatment with Test Article

Treatment with inhaled seralutinib or vehicle, RAP-011 or mIgG2A was as described in Table 2.

TABLE 2

| Group | N | State | Treatment | Dose |
|---|---|---|---|---|
| 1 | 5 | Healthy | — | — |
| 2 | 7 | PAH | Vehicle-Treated | Vehicle Inh (15 mg/kg) BID + |
| | | | Control | mIgG (2x/week) |
| 3 | 7 | PAH | SER Only | SER (15 mg/kg) BID + |
| | | | | mIgG 5mpk SC (2x/week) |
| 4 | 7 | PAH | SOR only | SOR 5 mg/kg SC (2x/week) + |
| | | | | Vehicle Inh (15 mg/kg) BID |
| 5 | 7 | PAH | SER + SOR | SER (15 mg/kg) Inh BID + |
| | | | Combo | SOR (5 mg/kg) SC (2x/week) |

SER = Seralutinib;
SOR = Sotatercept (dosed as RAP-011, the rat analog of Sotatercept)
Inh = dosed by inhalation;
SC = dosed as subcutaneous injection

Briefly, treatment with the aerosolized Seralutinib or vehicle was administrated for 45 minutes BID for 4 weeks starting day 22 (as outlined in Table 1) at an estimated dose to 15 mg/kg/dose using a Vilnius Aerosol Generator connected to an inhalation tower. The powder concentration was continuously monitored by portable aerosol monitor. Mercer-style cascade impactor was used to perform aerodynamic particle size distribution measurements. RAP-011 or mIgG was administered at 5 mg/kg dose via SC injection twice a week for 4 weeks starting day 22 as described in Table 2.

### Hemodynamics, RV Hypertrophy and Echocardiography Measurements

An echocardiogram monitoring of the progression of the disease was carried out on Day 21 (before treatment) and on surgery day (Day 49) for all groups. The results of the echocardiogram, from Day 21, was used to randomize the animals to ensure that only animals with severe enough disease are selected and that there is a similar disease stage across all groups.

On day 49, after 4 weeks of treatment, end of study procedures was performed: mean pulmonary arterial pressure (mPAP) and right ventricle systolic pressure (RVSP) were measured via an intra-ventricular fluid-filled catheter from AD instruments. Mean pulmonary arterial blood pressure values by using the formula mPAP=Diastolic Pressure+(Systolic Pressure−Diastolic Pressure)/3. Pulmonary vascular resistance index (PVRI) was calculated using the following formula: PVRI=mPAP/Cl, where mPAP is mean pulmonary arterial pressure (mm Hg) and Cl is cardiac index, defined as Cardiac Output (ml/min) normalized to 100 g body weight. As part of the Fulton index, the heart was dissected to separate the right ventricle from the left ventricle with septum, and then weighed separately, Fulton index=Right Ventricle weight/Inter ventricular septum weight+Left ventricle weight).

### Effect of the Test Article (Recovery)

The effect of the test article was calculated using the following formula:

((vehicle group value−test article group value)/(vehicle group value−normoxic group value))

### Statistical Analysis

Data is presented as median with interquartile range. One-way ANOVA followed by Fisher's LSD test were performed in GraphPad Prism 10.0 on all experimental conditions, comparing treatment groups to either the healthy animals (normoxic control), or the sick animals (SuHx+Placebo and mIgG2A). Differences were considered significant when *p<0.05, **p<0.01, ***p<0.001, ****p<0.0001.

### Data

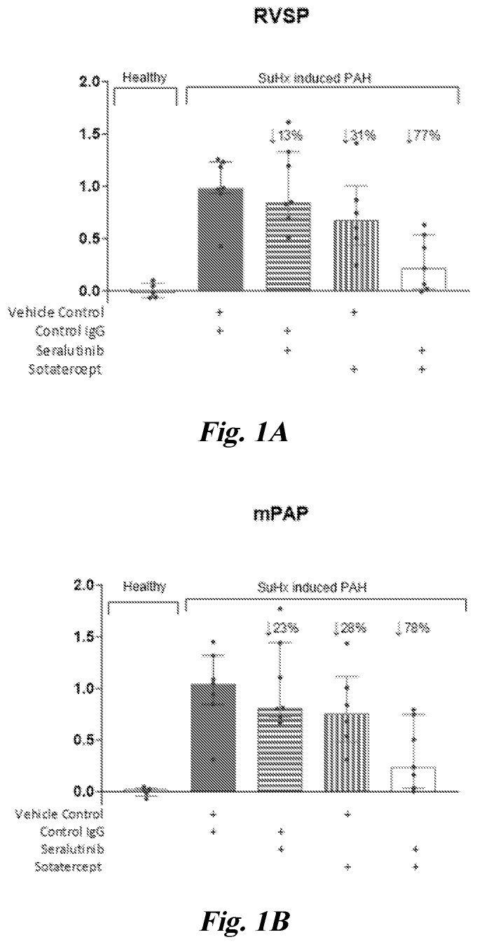

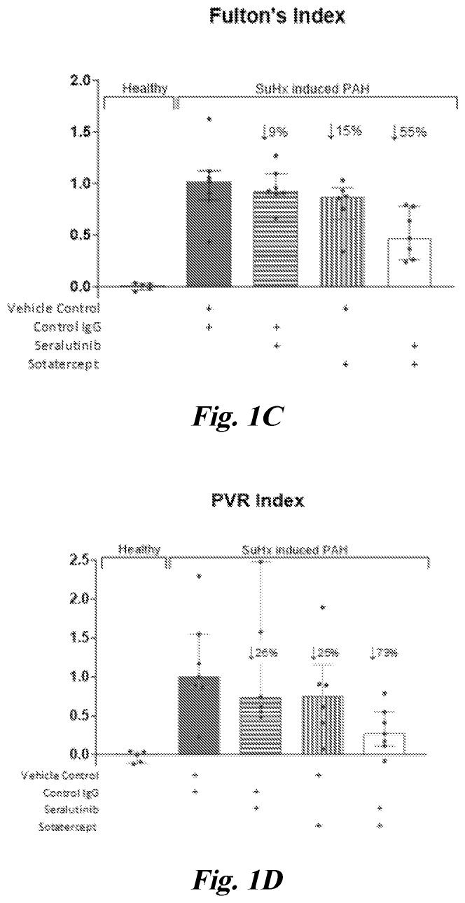

FIGS. 1A through 1D show that seralutinib inhalation plus sotatercept injection provided more than additive efficacy in this model of PAH. Referring to FIG. 1, data is presented the change in RVSP, mPAP, RV hypertrophy (Fulton's index) and PVR index for each treatment group (i.e., FIGS. 1A, 1B, 1C and 1D, respectively). The represented data was calculated by normalizing the median of each treatment with vehicle and normal controls. Parameter value=(Value−value of normal)/(value of vehicle−value of normal). n=5-7 per group. As shown by FIG. 1, the seralutinib+sotatercept treatment group showed more that additive benefit in four of the tested parameters (RVSP, mPAP, Fultons Index and PVR Index).

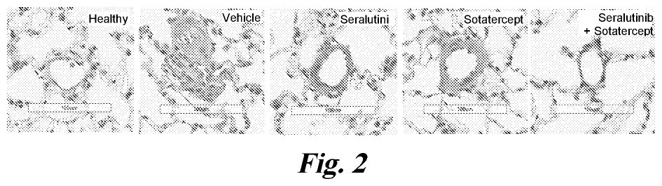

FIG. 2 shows representative image of pulmonary vessels from each treatment group. The vehicle controls animal which has significantly high mPAP and RVSP also has pulmonary vessels that were occluded, with neointimal proliferation and plexiform lesion, whereas the pulmonary vessels in healthy animals were not occluded. Seralutinib monotherapy, sotatercept monotherapy, and seralutinib+sotatercept combination therapy groups showed improvement in hemodynamic parameters were accompanied by non-occluded pulmonary vessels similar to the healthy group.

### Results

The Sugen-hypoxia rat model is a widely used and recognized model of severe PAH. Sugen 5416 (SU5416) is known to cause pulmonary endothelial cell apoptosis, when used at the working single dose of 20 mg/kg, combined to a three-week hypoxia (10% O₂) course like in this study, leads to severe PAH. Endothelial cell apoptosis, under hypoxic conditions, triggers endothelial cell proliferation in precapillary arteries and leads to the selection of an apoptosis-resistant subset of endothelial cells. This occurs in addition to the remodeling and constriction of vascular smooth muscle cells, furthering the development of PAH (Taraseviciene-Stewart, L., 2020). This occlusion of pulmonary arterioles leads to decreased blood flow and significantly raises the pulmonary arterial pressure (PAP), right ventricular systolic pressure and right ventricular hypertrophy.

In the present study, the vehicle controls animal groups receiving SuHx with seralutinib vehicle inhalation and sotatercept control IgG2A for 4 weeks, developed severe PAH, in 49 days. The vehicle controls (placebo inhalation+IgG2A) animals had statistically significant elevated RVSP (57.3 mmHg; IQR 65.7-55.7 mmHg) and mPAP (38.1 mmHg; IQR 43.3-34.2 mmHg) compared to normoxic control (RVSP 23.8 mmHg; IQR 26.95-22.4 mmHg and mPAP 18.2 mmHg IQR 18.7-17.2 mmHg; p<0.0001). Similarly, vehicle control animals also had significantly high RV hypertrophy measured using fulton's index (0.62; IQR 0.66-0.56) compared to healthy controls (0.26; IQR 0.26-0.24, p<0.0001).

Data represented is as calculated by normalizing the individual data point with vehicle (considered 1 and normal controls considered 0). Seraltuinib monotherapy treated animals had a 13, 23, 9 and 26% decrease, and sotatercept monotherapy animals had a 31, 28, 15 and 25% decrease, in RVSP, mPAP, Fulton's Index (RV hypertrophy measurement) and PVR Index, respectively. Whereas seralutinib plus sotatercept combination treatment group showed a 77% decrease in RVSP, a 78% decrease in mPAP, a 55% decrease in Fulton's Index, and a 73% decrease in PVR Index. Seralutinib plus sotatercept combination treatment showed significant decrease in RVSP, mPAP, Fulton's index and PVR Index compared to seralutinib monotherapy (p<0.001 for RVSP and mPAP and p<0.01 for Fulton's index and PVR index), and significant decrease in RVSP, mPAP and Fulton's index compared to sotatercept monotherapy (p<0.05 for RVSP, mPAP and Fulton's index.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

This application claims the benefit of priority to U.S. Provisional Application No. 63/396,899, filed Aug. 10, 2022, which application is hereby incorporated by reference in its entirety.

1. A method of treating pulmonary arterial hypertension (PAH), comprising administering to a subject in need thereof:
a therapeutically effective amount of a tyrosine kinase inhibitor or a pharmaceutically acceptable salt thereof; and
a therapeutically effective amount of a dimeric fusion protein comprising:
the extracellular domain of the activin type 2A (ACTR IIA) or the activin type 2B receptor (ACTR IIB); and
the Fc domain of human immunoglobulin G1 (IgG1).

2. The method of claim 1, wherein the tyrosine kinase inhibitor is a PDGF receptor inhibitor or a pharmaceutically acceptable salt thereof, a CSF1R receptor inhibitor or a pharmaceutically acceptable salt thereof, a c-KIT kinase inhibitor or a pharmaceutically acceptable salt thereof, or a combination thereof.

3. The method of claim 1, wherein the tyrosine kinase inhibitor is Acalabrutinib or a pharmaceutically acceptable salt thereof, Afatinib or a pharmaceutically acceptable salt thereof, Alectinib or a pharmaceutically acceptable salt thereof, Avapritinib or a pharmaceutically acceptable salt thereof, Axitinib (Inlyta®) or a pharmaceutically acceptable salt thereof, Baricitinib or a pharmaceutically acceptable salt thereof, Binimetinib or a pharmaceutically acceptable salt thereof, Bosutinib (Bosulif®) or a pharmaceutically acceptable salt thereof, Brigatinib or a pharmaceutically acceptable salt thereof, Cabozantinib or a pharmaceutically acceptable salt thereof, Capmatinib or a pharmaceutically acceptable salt thereof, Ceritinib or a pharmaceutically acceptable salt thereof, Cobimetinib or a pharmaceutically acceptable salt thereof, Crizotinib or a pharmaceutically acceptable salt thereof, Dacomitinib or a pharmaceutically acceptable salt thereof, Entrectinib or a pharmaceutically acceptable salt thereof, Erdafitinib or a pharmaceutically acceptable salt thereof, Erlotinib (Tarceva®) or a pharmaceutically acceptable salt thereof, Fedratinib or a pharmaceutically acceptable salt thereof, Fostamatinib or a pharmaceutically acceptable salt thereof, Gefitinib or a pharmaceutically acceptable salt thereof, Gilteritinib or a pharmaceutically acceptable salt thereof, Ibrutinib or a pharmaceutically acceptable salt thereof, Imatinib (Gleevec®) or a pharmaceutically acceptable salt thereof, Lapatinib or a pharmaceutically acceptable salt thereof, Larotrectinib or a pharmaceutically acceptable salt thereof, Lenvatinib or a pharmaceutically acceptable salt thereof, Lorlatinib or a pharmaceutically acceptable salt thereof, Midostaurin or a pharmaceutically acceptable salt thereof, Neratinib (Tasigna®) or a pharmaceutically acceptable salt thereof, Osimertinib or a pharmaceutically acceptable salt thereof, Pazopanib (Votrient®) or a pharmaceutically acceptable salt thereof, Pemigatinib or a pharmaceutically acceptable salt thereof, Pexidartinib or a pharmaceutically acceptable salt thereof, Ponatinib or a pharmaceutically acceptable salt thereof, Regorafenib or a pharmaceutically acceptable salt thereof, Ripretinib or a pharmaceutically acceptable salt thereof, Ruxolitinib or a pharmaceutically acceptable salt thereof, Selpercatinib or a pharmaceutically acceptable salt thereof, Selumetinib or a pharmaceutically acceptable salt thereof, Seralutinib or a pharmaceutically acceptable salt thereof, Sorafenib or a pharmaceutically acceptable salt thereof, Sunitinib (Sutent®) or a pharmaceutically acceptable salt thereof, Tofacitinib or a pharmaceutically acceptable salt thereof, Trametinib or a pharmaceutically acceptable salt thereof, Tucatinib or a pharmaceutically acceptable salt thereof, Upadacitinib or a pharmaceutically acceptable salt thereof, Vandetanib or a pharmaceutically acceptable salt thereof, Zanubrutinib or a pharmaceutically acceptable salt thereof, or a combination thereof.

4. The method of claim 1, wherein the tyrosine kinase inhibitor is Seralutinib or a pharmaceutically acceptable salt thereof.

5-7. (canceled)

8. The method of claim 1, wherein the dimeric fusion protein is Sotatercept.

9. (canceled)

10. The method of claim 1, wherein the subject is receiving stable background therapy for pulmonary arterial hypertension.

11. The method of claim 10, wherein the stable background therapy is monotherapy, double therapy, triple therapy, or quadruple therapy.

12. The method of claim 10, wherein the stable background therapy comprises an endothelin-receptor antagonist, a phosphodiesterase-5 (PDE-5) inhibitor, a prostacyclin analogue, a prostacyclin-receptor agonist, a soluble guanylate cyclase stimulator, or a combination thereof.

13. The method of claim 1, further comprising administering to the subject an endothelin-receptor antagonist, a phosphodiesterase type 5 (PDE-5) inhibitor, a prostacyclin analogue, a prostacyclin receptor agonist, a soluble guanylate cyclase stimulator, or a combination thereof.

14-25. (canceled)

26. The method claim 1, wherein the PAH is mild or moderate PAH.

27. The method of claim 1, wherein the PAH is moderate PAH.

28-29. (canceled)

30. The method of any one claim 4, wherein Seralutinib or a pharmaceutically acceptable salt thereof is administered by inhalation.

31-32. (canceled)

33. The method of claim 8, wherein Sotatercept is administered by injection.

34. The method of claim 8, wherein Sotatercept is administered by subcutaneous injection.

35-36. (canceled)

37. The method of claim 1, wherein the therapeutically effective amount of the tyrosine kinase inhibitor or a pharmaceutically acceptable salt thereof is administered prior to the administration of the therapeutically effective amount of the dimeric fusion protein.

38. The method of claim 1, wherein the therapeutically effective amount of the tyrosine kinase inhibitor or a pharmaceutically acceptable salt thereof is administered simultaneously with the administration of the therapeutically effective amount of the dimeric fusion protein.

39. The method of claim 1, wherein the therapeutically effective amount of the tyrosine kinase inhibitor or a pharmaceutically acceptable salt thereof is administered subsequent to the administration of the therapeutically effective amount of a dimeric fusion protein.

40. A method of treating pulmonary arterial hypertension (PAH), comprising administering to a subject in need thereof:
a therapeutically effective amount of Seralutinib or a pharmaceutically acceptable salt thereof; and
a therapeutically effective amount of Sotatercept.

41. A kit comprising:
one or more doses of a therapeutically effective amount of a tyrosine kinase inhibitor or a pharmaceutically acceptable salt thereof; and
one or more doses of a therapeutically effective amount of a dimeric fusion protein comprising:
the extracellular domain of the activin type 2A (ACTR IIA) or the activin type 2B receptor (ACTR IIB); and
the Fc domain of human immunoglobulin G1 (IgG1).

42-83. (canceled)

84. The kit of claim 41 comprising:
one or more doses of a therapeutically effective amount of Seralutinib or a pharmaceutically acceptable salt thereof; and
one or more doses of a therapeutically effective amount of Sotatercept.
